(19) United States
(12) Patent Application Publication
(10) Pub. No.: US 2002/0090614 A1
(43) Pub. Date: Jul. 11, 2002

(54) Direct measurement method for gene expression using single chain antisense array

(76) Inventors: Jia Zhang, Glendor, CA (US); Kai Li, Glendor, CA (US); Yunshi Zhang, (US); Xu Zhang, (US)

Correspondence Address: Raymond Yat Chiu Chan, 1050 Oakdale Lane, Arcadia, CA, 91006, US

(21) Appl. No.: 09/758,601

(22) Filed: Jan. 9, 2001

(51) Int. Cl. C12Q 1/68; C07H 21/04

(52) U.S. Cl. 435/6; 536/24.3

(57) ABSTRACT

A method for direct measurement of multiple gene expressions at RNA level is provided. Gene product RNA isolated from biological sample is directly used to hybridize with prelabeled single chain antisense probes immobilized on a solid support. As compared to the indirect assay of cDNA, the subject method is reliable to assay all types of RNA including ribosomal, transfer, messenger, and ribozyme RNAs, as well as mRNA with partial degraded. This invention finds use in assay for multiple gene expressions, especially when higher sensitivity and accuracy is required such as for those of minor changes and those of less abundant RNA.

### CROSS REFERENCE OF DISCLOSURE DOCUMENT

This non-provisional application is based on a DISCLOSURE DOCUMENT NO. 477186 entitled “Arrays for assay multiple gene expression at protein and mRNA levels”.

### BACKGROUND OF THE PRESENT INVENTION

1. Field of Invention

The invention relates to a measurement method of RNA, and more particularly to a method of direct measurement of RNA using prelabeled single chain antisense nucleotides of DNA or of RNA on a solid support to hybridize with RNA, and then digested with single chain specific nucleases. The probe protected by forming DNA:RNA or RNA:RNA duplex on array quantitates the target gene in a biological sample.

2. Description of Related Arts

There are some milestones in modern genetic technologies related to mRNA assay, such as Northern blotting, ribonuclease protection assay, and recently developed gene arrays.

Northern blotting is the typical method used for quantitative analysis of mRNA and it is still widely used by many laboratories. The advantage of Northern blotting is that it is simple, relatively easy and less expensive. However, Northern blotting is only able to check one gene per time. Ribonuclease protection assay is widely used because of its high sensitivity and capability of assaying more than one gene at a time. But ribonuclease protection assay is less powerful as compared to the recently developed gene chips which can assay thousands of genes at the same time. The principle of currently available gene chips will be described below.

In recent years, several methods targeting the assay of multiple gene expressions at the mRNA level have been invented. The common principle and main steps of the commercially available gene arrays, including cDNA array and polynucleotide arrays, are the following:

i). in vitro reverse transcription from mRNA into cDNA;

ii). Labeling of the in vitro reverse transcribed cDNA;

iii). hybridization of the labeled cDNA with gene array.

These commercial arrays possess some limitations in accuracy and sensitivity. First, these arrays assume that the labeled cDNA could realistically represent the expression level of mRNA. The cDNA is an ideal alternative used in gene assay but it is never the same as the mRNA itself in both the amount and the ratio between different kinds of genes. Secondly, the requirement of labeling for the in vitro transcribed cDNA decreases the sensitivity of the current available arrays since the labeling process is incomplete. The portion of the unlabeled cDNA does not only count in the decrease in assay sensitivity, but it also works as the competitor to the labeled ones during the hybridization procedure, thereby doubling the loss in sensitivity of the related assay. Thirdly, the strategy of using in vitro transcribed cDNA does not accurately work for partially degraded mRNA, and does not work at all for the mRNA with poly A tail degraded. Finally, the strategy of using in vitro reverse transcribed cDNA does not work for those types of RNAs that do not possess poly A tail such as transfer RNA, ribosomal RNA, and ribozyme genes; nor it works for mRNA after partially degraded at its 3′ terminal.

Compared to the Northern blotting and ribonuclease protection assay, currently available gene array provided the advantage of being able to measure multiple genes simultaneously. But, this gain was obtained with the sacrifice in sensitivity and specificity. As the classical method for RNA assay, Northern blotting and ribonuclease protection assay are still the best techniques and direct measurement of RNA for single gene expression assay. Ribonuclease protection assay was recently used for assaying several gene products simultaneous in situations where the target gene products are of different sizes. Clearly, indirect measurement of mRNA, the principle employed in the above mentioned gene arrays, cannot meet the rapidly increased demand for directly assaying multiple gene expressions.

The indirect analysis of multiple gene expression is well known as not being sensitive enough in gene profiling of many physiological and pathological processes, such as embryo development, cancer, hypertension, cardiac hypertrophy and heart failure. Although the employment of the currently available gene array has revealed some promising results in the situations mentioned above, it is far from understanding the detailed molecular mechanisms. There is a demand for more sensitive and direct method for multiple gene expression assays. In situations minor change in gene expression or gene targets are less abundant, dissecting the molecular mechanisms requires the application of more sensitive assay.

U.S. Pat. Nos. 6,004,755; 6,040,138; 6,087,102 also describe methods and new strategies for gene array preparations. Other references providing a review of technologies related to mRNA assay and microarray include:

(i) Current Protocols in Molecular Biology: chapters 4: pp 4.2.1-4.2.8; and 15: pp 15.1.1-15.1.5. John Wiley & Sons, Inc.

(ii) Molecular Cloning, A laboratory Manual, 2^{nd} edition, Cold Spring Harbor Laboratory Press 1989: Chapters 7: pp 7.6-7.15;10: pp 10.22-10.24; and 14 pp 14.18-14.21.

(iii) Clontech Catalogue, Chapter 4: cDNA array; 1998/1999 pp 48-55.

(iv) Promega Catalogue, S1 nuclease, p 3.17, 2000

### SUMMARY OF THE PRESENT INVENTION

The main object of the present invention is to develop a direct measurement for multiple gene expression assays. By using a new strategy, the method in the subject invention labels single chain antisense DNAs or RNAs, immobilize them on a solid supporter, hybridized with target RNA, and then remove the unhybridized probes by single chain specific enzyme, thereby inheriting the advantages of classical Northern blotting, ribonuclease protection assay, and the modern cDNA arrays. The method of the invention greatly improved the sensitivity and reliability of multiple gene assays and eliminated the noise from the non-specific binding between labeled gene targets and the surface of solid support in other assay methods.

The increased accuracy and sensitivity of this method is benefited from the employment of labeled single chain probes and single chain specific enzyme digestion. Single chain specific nuclease is used to digest the unhybridized prelabeled probe printed or blotted on the solid support, a principle similar to ribonuclease protection assay. The protected prelabeled probes quantitate the corresponding RNA targets. The method of the present invention avoids the incomplete enzymatic reactions of in vitro transcription and labeling. The efficiency of in vitro transcription is variable depending on the enzyme or the kit used from different companies, ranging from 10% to 60%. Overall, the present methods raise the sensitivity and specificity of gene chip to or even higher than the level of the ribonuclease protection assay. In fact, there is no signal loss from running a gel as occurred in ribonuclease protection assay. Clearly, the present invention provides a direct, reliable, and sensitive method for multiple gene expressions at RNA level.

The present invention possesses numerous advantages over the prior art. One of the major advantages is the direct measurement of RNA by hybridizing with prelabeled gene arrays. This strategy avoids those incomplete processes of in vitro manipulations of gene products before analyzing them; it thus provides novel methods for direct measurement of multiple gene expressions of RNAs, instead of in vitro reverse transcribed cDNA. The second major advantage is a more accurate assay of mRNA including those of partially degraded mRNA, which is very useful in situations where some specific mRNAs are in rapid degradation processes. Another major advantage is that this invention has increased sensitivity as compared to other multiple gene expression assays. This feature is especially useful in the analysis of low abundant mRNA targets and in circumstances when physiological and pathological effects are caused by minor change in the expression of some specific genes. The final advantage is that in addition to mRNA this method is effective to assay all other types of RNA such as transfer RNA, ribosomal RNA, and ribozyme RNA.

### BRIEF DESCRIPTION OF THE DRAWINGS

FIGS. 1A and 1B illustrate the strategy of using prelabeled single chain antisense probes for direct measurement of gene expression at RNA level.

FIG. 2 illustrates the single chain antisense DNA production from unidirectional primer extension.

FIG. 3 illustrates a representative result of gene expression assay using prelabeled single chain antisense probes.

### DETAILED DESCRIPTION OF THE PREFERRED EMBODIMENT

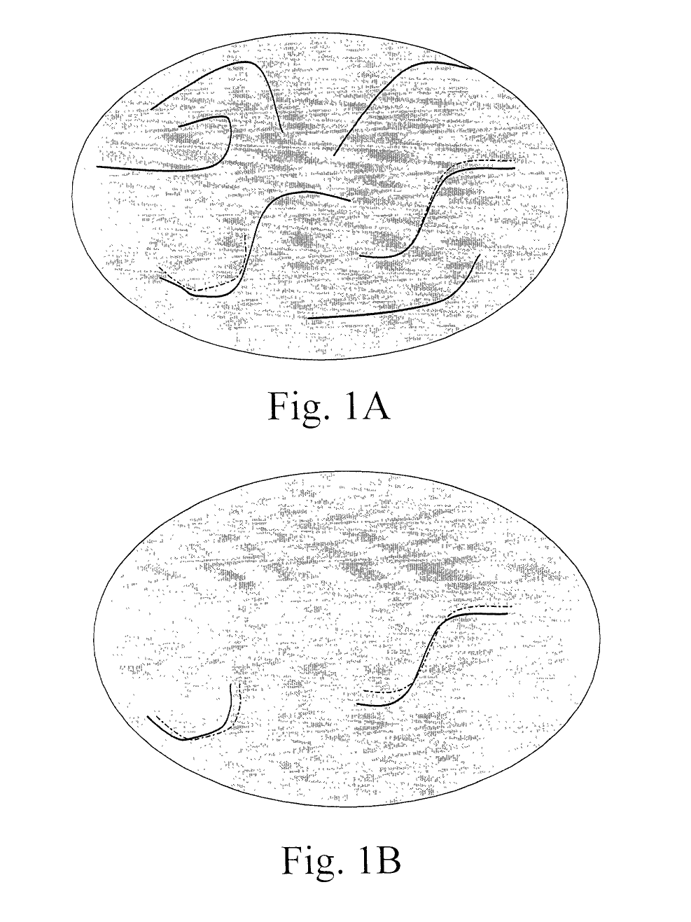

Referring to FIGS. 1 to 3, the present invention provides a method of direct measurement of gene expression using single chain antisense array.

FIG. 1 depicts the strategy of using prelabeled single chain antisense probes for direct measurement of gene expression at RNA level, wherein solid lines represents the prelabeled antisense probes immobilized on an array. Panel A: An array with prelabeled antisense probes is hybridized with mRNAs directly isolated from biological sample. Dashed line represents the mRNA. DNA/RNA duplexes are formed between some probes and their complementary mRNAs. Panel B: The array is digested by a single chain specific nuclease S1. All unhybridized probes are removed and only probes or the part of the probe formed DNA:RNA duplex survived from S1 digestion. The protected prelabeled probes provide quantitative information for gene expression.

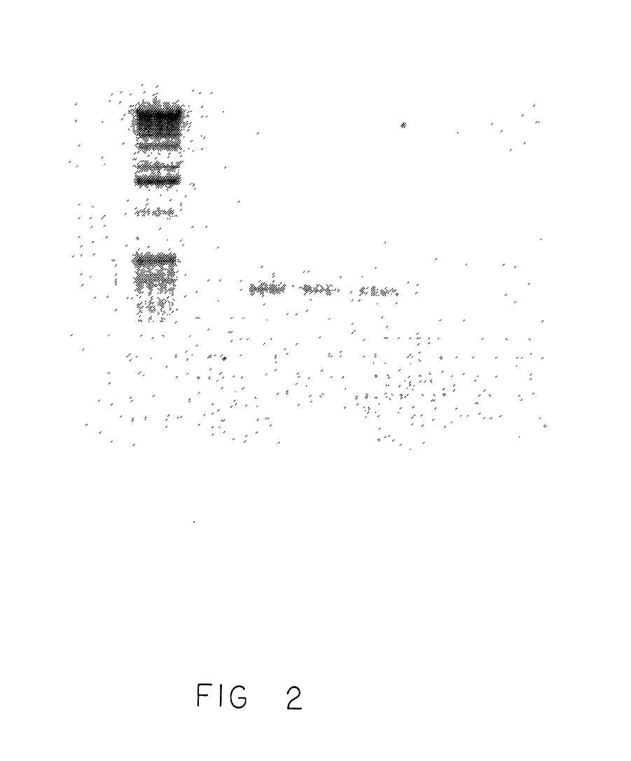

FIG. 2 depicts the single chain antisense DNA production from unidirectional primer extension. The rat monocyte chemoattractant protein-1 (MCP-1), was amplified using Kpn I digested pGL3-MCP1_{—}500 plasmid as template. An equal amount of template of 15 ng per reaction was used in a total of 0.2 ml reaction solution and PCR products were precipitated before loading. Lane 1 shows 300 ng of the 1 kb double strand DNA marker where the first bright band is 500 bp. Lanes 2 to lane 6 shows the unidirectional extension products from 100, 80, 60, 40, and 20 cycles using Deep Vent polymerase. Unidirectional primer extension condition: 95° C. for 30 seconds, followed by 65° annealing for 5 seconds, and extension at 72° C. for 25 seconds. Cycles were different as mentioned above. The long half-life of DeepVent polymerase allowed the unidirectional extension to work at very high number of cycles.

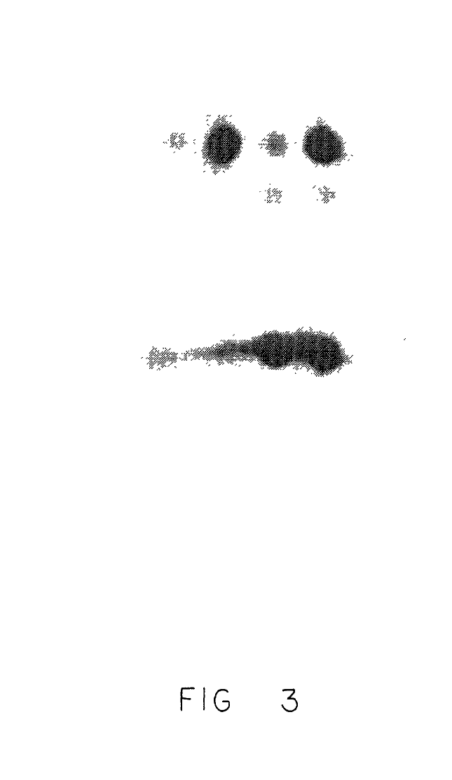

FIG. 3 depicts a reprehensive result of gene expression assay using prelabeled single chain antisense probes. Hybridization between rat kidney total RNA and a solid support containing digoxigenin prelabeled single chain antisense probe to MCP-1 partially protected the MCP-1 probe from S1 digestion. On the same array, housekeeping genes GAPDH and beta-actin showed much stronger signals, which were used as internal control for calibration.

A plurality of terminologies are used in the detail description of the present invention, which are defined as follows:

PCR refers to polymerase chain reaction. A variety of PCR techniques have been developed and primers used in PCR are usually 2 (one pair) or more than 2.

Label refers to the incorporation of an easily detectable reagent to nucleic acids, either the immobilized nucleic acids on solid support or the nucleic acids in the solution. In this invention, the label specifically refers to the incorporation of either isotopic or non-isotopic reagents into the single antisense DNA or RNA used for array preparation.

Probe refers to the prelabeled materials used to detect gene target by hybridization. Gene probe can be any chemical with specific binding activity to gene target. A probe can be either in hybridization solution or immobilized on a solid supporter but the term probe used in this invention only refers to the prelabeled antisense nucleic acids immobilized on a solid supporter.

Gene target refers to the gene products to be analyzed, which mirrors gene probe here.

Nucleic acid refers to either DNA (deoxyribonucleotidyl acid) or RNA (ribonucleotidyl acid).

DNA refers to deoxyribonucleotidyl acid. As the genetic material, DNA molecule can be replicated to more molecules and be transcribed to RNA.

cDNA refers to complementary DNA as compared to messenger RNA. cDNA is prepared by in vitro reverse transcription from mRNA.

RNA refers to ribonucleotidyl acid, including transfer RNA, ribosomal RNA, and messenger RNA. RNA is usually transcribed from DNA but it also can be reverse-transcribed to DNA as seen in retroviruses.

mRNA refers to messenger RNA. Messenger RNA is transcribed from DNA and it is used as a template or model for the synthesis of protein. In experimental condition, mRNA can be used to in vitro reverse transcribe into complementary DNA for the purposes of genetic engineering and for gene expression assays such as used in microarray.

Antisense refers to the genetic sequences complementary to messenger RNA that is defined as sense chain. The rule of the complementary is A to T (U) and C to G.

Northern blotting refers to the typical method for quantitative measurement of mRNA by hybridizing labeled probe with immobilized RNA sample on membrane, where the probe can be labeled using either isotopic or non-isotopic method.

In vitro transcription refers to the transcription procedure from DNA to RNA carried on in a tube instead of inside a cell. DNA dependent RNA polymerase is required in this process. In vitro transcription is widely used to prepare single chain RNA probe.

Ribonuclease protection assay refers to the most sensitive and reliable method for RNA assay, in which hybridization reaction is done in solution phase and unhybridized probe is removed by ribonuclease digestion and the RNA:RNA or RNA:DNA duplex is further analyzed. Classical application of this method was used for single gene expression assay. Recently, this method was employed for analysis of more than one gene expression if the differences in size were large enough for gel separation.

Direct measurement of gene expression refers to the methods of assaying gene products directly, such as Northern blotting and ribonuclease protection assay for mRNA and Western blotting and enzyme-linked immunosorbent assay (ELISA) for protein. Gene assay using materials from in vitro transcription or PCR amplification are not direct or less direct.

The present invention provides a direct method for the quantitative analysis of multiple gene expression using prelabeled single chain antisense probes immobilized on a solid support to hybridize to RNA sample and then digested by single strand specific nuclease to remove the unhybridized probes.

Prior to this invention, polynucleotide arrays and cDNA arrays are used for multiple gene expression assays. However, the reliability and sensitivity are quite different between single gene assays and multiple gene assays. At mRNA level, multiple gene expression assays are less reliable and less sensitive than conventional method such as Northern blotting and ribonuclease protection assays. Indirect assays for multiple gene expression are widely used with currently available arrays. These arrays are useful for assaying most of the abundant genes but not sensitive for less abundant gene products. Among the techniques used for gene expression assay, ribonuclease protection assay is still the most sensitive one. Considering sensitivity and reliability alone, ribonuclease is better than Northern blotting, and Northern blotting is better than gene array.

Compared to conventional direct gene assay of Northern blotting and ribonuclease protection assay, the present invention extends the application of direct measurement of mRNA to more than 10, preferably to more than 100 genes, and most preferably of more than 1000 gene targets. The method in this invention simplifies the procedures of the assay, rendering it more accurate, more sensitive, less equipment dependent, and less expensive.

In the subject method, prelabeled single strand nucleotide probes is coated on a solid supporter. The target nucleic acids, preferably message RNA, which is directly isolated from biological samples are then hybridized with the prelabeled probes to form double strand duplex or dimer. The dimer formation from the hybridization protects the prelabeled probe from the subsequent digestion using single strand specific nucleases. This pattern is similar to the well-known ribonuclease protection assay except the principle is reversed. In this way, each positive signal on the solid support is precisely and proportionally derived from the target sample, since only the probes which form a duplex with the target gene can be protected from single chain specific nuclease digestion. This invention keeps the inheritance of high sensitivity from ribonuclease protection assay and extends the application for multiple gene expression assays to the field of gene chip. The subject method finds use in a variety of applications especially in cases of assaying multiple gene expression of RNAs in low abundance.

In the subject method, the prelabeled single chain probes are covalently linked to the solid supporter using a variety of methods known in the art. The length of the prelabeled single chain antisense probes are preferably between 200 and 800 nucleotides. For mRNA targets longer than 1000 nucleotides, more than one region will be used to prepare their single chain probes in the array.

As compared to the current commercial available gene chip for indirect assay of multiple gene expressions, the major advantage of the subject method is the direct measurement of RNA including mRNA, instead of measurement of the in vitro transcribed cDNA. The mRNA in the sample that is contacted with the array during the assay is directly and precisely detected by forming DNA:RNA or RNA:RNA duplexes in which prelabeled probes are protected from single chain specific enzyme digestion. The direct assay of mRNA avoids the variations caused during in vitro transcription and in vitro labeling of the cDNAs.

Another advantage of the subject method is that this invention is able to detect partially degraded mRNAs. In contrast, using cDNA as an estimation of gene expression in the currently available gene chips is not possible to detect any partially degraded mRNAs. This unique feature is especially useful in situations when rapid degraded mRNAs are to be assayed.

It is to be understood that the invention is not limited to the particular embodiments of the invention described below, or to the examples of the invention included below.

I. Coating solid support with the prelabeled single strand antisense probes

A).1. Preparation of single chain DNA probe using unidirectional primer extension

In a method of preparing single chain DNA, unidirectional primer extension is employed using Deep Vent polymerase from New England Biolabs Inc (Beverly, Mass. 01915-5599). The templates used for unidirectional primer extension are double strand DNA, including plasmids with cDNA or genomic DNA of target genes and PCR products of target genes. Techniques related to the template preparation include methods for cDNA library, for genomic library, for reverse transcription, and for PCR (see details in Current Protocols in Molecular Biology, John Wiley & Sons, Inc).

Deep Vent polymerase is chosen mainly because its long half-life (about 8 hours) at 95 degrees centigrade. This feature of long half-life allows us to run more than 100 cycles for the unidirectional primer extension. Different polymerases have different characteristics, such as hot start polymerase from Promega (Madison, Wis. 53711-5399) can effectively eliminate most of the nonspecific PCR products by initiating polymerase enzymatic activity following 15 minutes preheating at 95 degree centigrade. In conventional PCR that contains 1 pair or more than one pair of primers, its products increase exponentially with reaction cycles, namely, products=2ⁿ×template where n is the number of cycles the PCR runs). Unidirectional primer extension only uses one primer, the antisense primer. The amplification of the product in a unidirectional primer extension equals the times of the cycles it runs, namely product=n×template. Therefore, the run cycle is crucial for unidirectional primer extension.

In a particular embodiment, the template is prepared by conventional PCR. Conventional PCR is run using one pair of primers, sense primer and antisense primer. This is done in two specific cases: a specific target gene is over 70 percent homogenous with another gene due to gene redundancy, and no unique site is available to cut this gene to provide a more specific single chain DNA probe. In this case, the antisense primer used for conventional PCR is about 500 bp, preferably 1 kb, down stream of the antisense primer that is to be used for unidirectional primer extension. Spatial separation of the two antisense primers permits a better gel purification. Products from the unidirectional primer extension are either gel purified or exonuclease III digested to eliminate the double strand DNA template.

Most gene probes are amplified from plasmids. Which segment of the target gene used to be the gene probe is controlled in two ways. The first control is the antisense primer, which determines the beginning of the single chain DNA probe. The antisense primer varies in length from 20 bases to 30 bases according to the GC contents of the primer: higher GC contents require a shorter primer and lower GC contents require a longer primer. The second control is the restrictive endonuclease used, which cuts the 5′ end of the plasmid. In case poly T is required, antisense primer is tagged with a poly T tail ranging from 5 to 50 nucleotides. Unidirectional primer extension amplifies target gene in one direction. It starts from where the antisense annealed and terminates at the site that restrictive endonuclease cuts.

The reaction protocol of the unidirectional primer extension is described in the following 3 steps with cycling. Step 1 for denature is set at 95 degree centigrade for 5 minutes. Step 2 for annealing is set at 58 to 68 degree centigrade for 5 to 30 seconds according to the length of the primer. Step 3 for extension is set at 72 degree centigrade for 30 seconds for 1 kb product and the extension time is proportionally increased at a base of 1 minute per kb. The cycle is set between 80 and 120 based on the yield of the specific gene target.

A). 2. Another strategy for preparing single antisense chain of DNA is to use two steps of conventional PCR.

Specific cDNA libraries, most of them such as human cDNA libraries, rat cDNA libraries, and mouse cDNA libraries, commercially available now, are used to subclone specific genes. One pair of primers is used to obtain the target template, then unidirectional primer extension using a single primer is used for probe labeling. This design is similar to the conventional nested PCR but the difference is that one and a half pair primers are used instead of two primers.

For both methods of A.1 and A.2, the double strand template used for unidirectional primer extension will be removed by exonuclease III digestion. Since exonuclease III only requires a substrate with a recessive or blunted 3′ end, the template must be enzymatically modified to have the right pattern of 3′ end for the sense chain of the template and to have an extrude 3′ end for the antisense chain of the template.

A). 3. Single strand antisense DNA probe can be synthesized using conventional PCR with exonuclease III digestion. The concentration of exonuclease is about 150 U/pMol susceptible 3′ terminals. The temperature and duration of digestion is upon to the length of the probes to be prepared. The conventional PCR products can be either labeled during the PCR process or labeled after exonuclease III digestion.

The product from conventional PCR is a double strand DNA. The exonuclease III requires a substrate having a recessive or blunted 3′ terminal. This enzymatic property can be satisfied during the preparation of PCR template, by adding a 3′ recessive restrictive site at the downstream of the probe region. For example, when subcloning plasmids for the preparation of antisense probes, vectors will have polylinker sites including Hind III, Xho I, or BamH I at the its 3′ end as the products from the digestion of these restriction enzymes are good substrates of exonuclease III. For short probes, exonuclease III digestion is carried out at 30° C. For long probes, 37° C. is used for exonuclease III digestion. The enzymatic activity is varied at different temperature: it removes about 250 nucleotides per minute at 37° C. and about 120 nucleotides per minute at 30° C. Low temperature is recommended for exonuclease III digestion as a higher specificity of cutting one strand of the double strand DNA.

Another way to satisfy the requirement of exonuclease III is through primer design. The above mentioned restrictive enzyme site of Hind III, Xho I, or BamH I is flanked at the 5′ end of the antisense primer. This strategy and the one mentioned above is very useful but their use is based on the condition that there is no such restrictive sites within the sequence or such site is point mutated before preparing the probes.

A). 4. Single strand antisense DNA probe can be synthesized using M13 or phagemid DNA. Probes longer than 200 nt prepared with this method are purified by electrophoresis. Isolation and purification of shorter probes can be done with alkaline chromatography.

A). 5. Strategy for preparing prelabeled single antisense chains of RNA: in vitro transcription uses the bacteriophage promoters such as T7, T3, and SP6 for template preparation and the corresponding RNA polymerase for in vitro transcription. Either isotopic or non-isotopic RNA probes are prepared based on substrates added. In vitro transcribed RNA probes are then treated with RNase-free DNase I to remove the DNA template. Gel or column purified RNA probes are saved for array preparation.

B). Prelabeling of single strand antisense probes

B.1. Single strand antisense probes are labeled during the process of unidirectional extension. Small amounts of chemically modified deoxynucleotides (dNTP), such as isotope labeled, fluorescent labeled, digoxigenin or biotin labeled dNTPs, are proportionally mixed with regular dATP, dTTP, dCTP, and dGTP at specific concentrations. In order to make the labeling procedure easier to perform and not interfere with the subsequently hybridization procedure, nucleotide base of purines or pyrimidines may be replaced by their derivatives with similar or same hybridization characteristics. This is more frequently seen in the methods of using digoxigenin and biotin labeling.

The reaction conditions used to prepare labeled single strand antisense probes are described in detailed in section A previously. Labeled probes can be purified using Qiagen DNA purification columns according to the manufacturer's directions. The yield of the unidirectional extension for each probe is evaluated before being applied to an array production.

B.2. Prelabeled primers are used in unidirectional reaction. This method is mainly used for digoxigenin and biotin labeling but it is also good for isotopic labeling and fluorescent labeling.

B.3. Prelabeled RNA probes are prepared during the in vitro transcription as described above.

B.4. Where short targets are to be detected, e.g. targets shorter than 100 nucleotides and usually shorter than 300 nucleotides, chemically synthesized prelabeled probes with variable length are directly used as the single antisense probe for array preparation.

C). Precoating and coating a solid supporter

The solid support of the RNA array is precoated with milk protein, poly T ranging from 5 to 50, or mixture of protein and poly T. Milk protein precoating renders NH2 available to covalently link the single chain antisense probe to the solid supporter. During preparation of the single chain antisense probe, the primer to be used is modified with specific moieties such as sulfosuccinimidyl substrate. This modification or a similar modification allows the antisense single chain to form a covalent bond by reacting with the amino groups. This reaction is fast and complete with a pH higher than 8. This reaction is irreversible and the reaction product is not cleavable. After forming a covalent binding, the single chain antisense probe is immobilized on the solid supporter with its 5′ terminal. This type of immobilization is different from UV-cross linking used in conventional gene expression assays or cDNA gene arrays. During the following hybridization process, the antisense probe is completely free for mRNA to access. In addition, these probes are completely exposed to single chain specific nuclease in the digestion process. Immobilization by UV cross-linking partially prevents the formation of DNA:RNA and RNA:RNA duplexes and also decreases the efficiency of single chain specific nuclease. Similar to regular ribonuclease protection assay, the antisense single chains only have two fates: forming a duplex with mRNA or being digested by single chain specific nuclease.

Solid support precoated with poly T nucleotide renders a high-affinity binding to mRNA with poly A tails. The hybridization pattern between poly T nucleotide precoated on a solid support and the poly A tail of mRNA is temperature and hybridization buffer stringency dependent. The optimal hybridization condition is that it attracts most of the mRNA to the solid support and facilitates the specific duplex formation between the target gene products and said prelabeled single chain antisense probes.

Considering the requirement of assaying partially degraded RNA sample, one array can contain more than one type of coating pattern. The single chain probe coating can be mixed with previously UV linked probes in the same array. Another alternative is that a small amount of chemically modified dNTP(s) or NTP(s) with specific moieties such as sulfosuccinimidyl substrate will be used during the preparation of single chain antisense probe. Thus, a single array can be coated in several ways. One pattern of coating or the different combinations of the following 3 coating can be applied to any array: one-point covalent linked probes, several-sites covalent linked probes, and multiple sites UV-cross linked probes.

In a particular embodiment of UV-cross link, single chain antisense probes with multiple nucleotides prelabeled require the labeled moieties to only reside in adenosine, cytidine, or guanodine, but generally not in thymidine nor uridine. S1 digestion is not able to remove the covalently cross-linked thymidine or uridine on solid support if baking or UV cross-link is applied. All of the other 3 types of base residues are not linked to solid support after cross-link. Since signals only come from labeled probes coated on the array, the subject invention eliminated those noises that are from the labeled gene products as seen in other gene expression methods, therein significantly increasing the signal/noise ratio.

When single chain antisense probes are prepared by primer extension or in vitro transcription, antisense primer is tagged with a poly T nucleotide ranging from 5 to 50. This poly T tag enforces the cross-linking between labeled probe and the solid support and increases the binding affinity of the solid support to mRNA through the interaction between poly T tags and poly A tails.

Chemical modification may alter the cross-link property of thymidine or of uridine, such as the case of digoxigenin modified uridine. Single chain probe labeled with digoxigenin with UV cross-linked on membrane can be digested by S1 nuclease.

Prelabeled probes can be blotted or printed on the subject array using either manual or automatic methods. In addition to the prelabeled probes complementary to target genes of interest, probes complementary to housekeeping genes of the same species, non-homogenous genes from other species, and artificially synthesized DNA with specifically designed random sequences are also spotted on the array for calibration and for quantitative analysis.

II. Hybridization

Prehybridization required in most other gene expression assays is usually not needed in this invention as the precoating treatment decreases the non-specific interaction between the solid supporter and reagents used in procedures of visualization and detection. The RNA targets used in this method are “cold” or not labeled, any non-specific binding between RNA targets and the array does not decrease the signal/noise ratio. Adding extra blocking reagents such as salmon sperm DNA into the hybridization buffer does not necessarily increase the signal/noise ratio.

A gene array is first incubated with hybridization buffer for 5 to 10 minutes at a temperature ranging between 55 and 68° C. depending on whether the array probes are single RNA chains or single DNA chains. The principle determining hybridization is whether the duplex is DNA:RNA or RNA:RNA; the former requires a higher hybridization temperature and the latter needs a lower hybridization temperature. Before hybridization, the RNA sample needs to be mixed with a hybridization buffer and denatured for 5 minutes at 95° C. The denatured RNA sample is then added and well mixed with the gene array for hybridization.

A one to four-hour hybridization is followed at a selected temperature between 50 and 68° C. For solid support precoated with poly T nucleotide, a touch-up hybridization pattern helps to abbreviate the hybridization duration and facilitate the binding efficiency between labeled single chain antisense probes and target mRNA. A touch-up hybridization starts at 50° C. for 10 to 30 minutes and then raises the hybridization temperature up to 55 to 68° C. The touch-up hybridization can be repeated to increase the hybridization efficiency. A DNA:RNA or RNA:RNA duplex formed during hybridization is stable in the hybridization buffer for 24 hours. Following hybridization, the gene array is washed and the un-hybridized probes are subjected to digestion with S1 nuclease.

III. Remove unhybridized probe using S1 nuclease digestion

S1 nuclease is a single strand specific endonuclease that digests both single stranded RNA and single stranded DNA. The principle of using S1 nuclease in this invention is similar to the use of ribonuclease in a ribonuclease protection assay: only the labeled probes hybridized with RNA will not be digested by S1 nuclease. Compared to ribonuclease protection assay, the present method allows assaying hundreds to thousands of genes in one assay.

Following hybridization, a 30-minute to two-hour digestion with S1 nuclease will follow. The final concentration of S1 nuclease is between 500-1000 units/ml and digestion is carried out at temperature between 37° C. and 45° C. Depending on the abundance of specific target genes and the requirement of sensitivity, the digestion procedure can vary in duration. The major point to choose an optimal duration of S1 nuclease is to minimize background and increase signal/noise ratio. This step will remove all or most of the unhybridized probes on the gene array, which then allows precise evaluation of the mRNA from the amount of probe based on their one to one pattern of hybridization.

IV. Visualization and detection of hybridized probe

Prior to visualization and detection, 3×5 minute wash is carried out using different stringencies of SSC. No extensive wash is required as the RNA targets are not hot as mentioned above. The resultant hybridization pattern(s) of labeled probes and target RNAs may be visualized or detected in a variety of ways. Additional visualization is only required for those labeling methods which are not directly detectable such as digoxigenin and biotin. Either chemiluminescent or fluorescent reagents are then applied to visualize the hybridized patterns. For fluorescent labeled probes, simple light stimulation is reached using a specific wave length.

The particular method of detection being chosen is based on the particular label of the probes printed or blotted on the solid support. Examples of detection methods include those for non-isotopic labeled probes such as fluorescence measurement, light emission measurement, and calorimetric measurement; and those for isotopic labeled probes such as autoradiography and scintillation counting. These detection methods are well known to those of skill in the art familiar to the particular signal producing system employed.

Following detection, the relative and absolute level of the gene target to be assayed is calculated using the housekeeping gene as one normalization parameter system and further calculated using the external unrelated genes as another normalization parameter system. In most situations, biological meaning comes from the comparison of the hybridization patterns between different samples.

This invention provides a direct, accurate, sensitive, quick, and simple technique for multiple gene expression assays. Neither in vitro reverse transcription from mRNA to cDNA nor in vitro labeling is required to the end user. RNA samples are directly used for hybridization using the new array claimed in the subject invention. The subject invention finds use in multiple gene expression assays, especially in cases requiring high sensitivity.

What is claimed is:

1. A method of direct measurement for gene expression of one or more genes, comprising the steps of:
(a) hybridizing a RNA preparation directly isolated from a biological sample to an array with prelabeled antisense single chain probes;
(b) removing unhybridized prelabeled probes from said array by a single chain specific enzyme digestion; and
(c) visualizing and detecting protected probes from said single chain specific enzyme digestion.

2. The method as recited in claim 1 wherein said prelabeled antisense single chain probes include antisense single chains of DNA and antisense single chains of RNA.

3. The method as recited in claim 2 wherein said antisense single chains of DNA are made by amplifying the target gene using unidirectional primer extension.

4. The method as recited in claim 2 wherein said antisense single chains of DNA are made by using two directional primer extension from which single chains of DNA are then prepared by enzyme digestion.

5. The method as recited in claim 2 wherein said antisense single chains of RNA are made by in vitro transcription.

6. The method as recited in claim 1 wherein said prelabeled antisense single chain probes comprises isotopic and non-isotopic labeling.

7. The method as recited in claim 1 wherein said hybridization is performed at temperature between 50 and 68° C.

8. The method as recited in claim 1 wherein said hybridization is performed in a touch-up pattern, which starts at low temperature for concentrating mRNA onto solid support and then switches to high temperature for specific duplex formation between labeled probes and target RNA.

9. The method as recited in claim 1 wherein said array with prelabeled antisense single chain is precoated with milk protein.

10. The method as recited in claim 1 wherein said array with prelabeled antisense single chain is precoated with poly T nucleotides.

11. The method as recited in claim 1 wherein said array with prelabeled antisense single chain is precoated with milk protein and poly T nucleotides.

12. The method as recited in claim 1 wherein said prelabeled antisense single chain probes are coated on a solid support by forming covalent link using UV cross-linking.

13. The method as recited in claim 1 wherein said prelabeled antisense single chain probes are coated on a solid support by forming covalent link using baking.

14. The method according to claim 1, wherein said prelabeled antisense single chain probes are coated on a solid support by forming a one site covalent link through a chemical reaction.

15. The method as recited in claim 12 wherein said prelabeled antisense single chain probe is tagged with a poly T tail at one terminal.

16. The method as recited in claim 13 wherein said prelabeled antisense single chain probe is tagged with a poly T tail at one terminal.

17. The method as recited in claim 14 wherein said solid support is precoated before coating said prelabeled antisense single chain probes, which is then used for covalent formation through one site linking through chemical reaction between one terminal of said prelabeled antisense single chain probes and said precoated solid support.

18. The method as recited in claim 1 wherein said prelabeled antisense single chain probes comprise fragments of between 1 and 5 different length antisense single chains of DNA for each individual gene target.

19. The method as recited in claim 1 wherein said prelabeled antisense single chain probes comprise fragments of between 1 and 5 different length antisense single chains of RNA for each individual gene target.

20. The method as recited in claim 1 wherein said array is in the format of macroarray.

21. The method as recited in claim 1 wherein said array is in the format of microarray.

22. The method as recited in claim 20 wherein said macroarray is a gene array for assaying less than 100 and preferably for less than 50 genes on a single array.

23. The method as recited in claim 21 wherein said microarray is a gene array for assaying more than 100 and preferably more than 1000 genes on a single array.

24. The methods as recited in claim 1 wherein said single chain specific enzyme digestion is carried by a single chain specific nuclease which is a family of nucleases that specifically breaks down a single chain of RNA and DNA.

25. A kit for use in a hybridization assay, comprising a set of gene arrays with prelabeled antisense single chain probes, a single chain specific enzyme, buffers, and regents for visualization and detection.
